(19) United States
(12) Patent Application Publication
(10) Pub. No.: US 2026/0315804 A1
(43) Pub. Date: Oct. 15, 2026

(54) IMPROVED FEEDBACK CONTROL OF NEURAL STIMULATION THERAPY

(71) Applicant: Saluda Medical Pty Limited, Macquarie Park, New South Wales (AU)

(72) Inventors: Dean Michael Karantonis, Macquarie Park (AU); Peter Scott Vallack Single, Macquarie Park (AU); Gerrit Eduard Gmel, Macquarie Park (AU); Daniel John Parker, Macquarie Park (AU); Samuel Nicholas Gilbert, Macquarie Park (AU)

(21) Appl. No.: 19/104,489

(22) PCT Filed: Aug. 18, 2023
(86) PCT No.: PCT/AU2023/050786

§ 371 Date: Feb. 18, 2025

(30) Foreign Application Priority Data
Aug. 19, 2022 (AU) 2022902371

(51) Int. Cl. A61N 1/36

(52) U.S. Cl. CPC A61N 1/36139; A61N 1/36062; A61N 1/36071

(57) ABSTRACT

An implantable device (100) to control the delivery (1110) of neural stimuli via stimulus electrodes (150) to a neural pathway of a patient (108) in order to evoke a neural response. Capture (1120) sensed signal windows to measure (1130) a first characteristic from a first captured signal window and a second characteristic from a second captured signal window. Analyse (1140) the first measured characteristic and the second measured characteristic to determine one or more feedback variables, to then adjust (1140) the plurality of stimulus parameters so as to maintain the one or more feedback variables at respective target values.

The present application claims priority from Australian Provisional Patent Application No 2022902371 filed on 19 Aug. 2023, the contents of which are incorporated herein by reference in their entirety.

### TECHNICAL FIELD

The present invention relates to neural stimulation therapy and in particular to improved feedback control of neural stimulation therapy when multiple measurements of neural responses are available.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Action potentials propagating along Aβ (A-beta) fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ (A-beta) fibres. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres. Approaches proposed for obtaining a neural response measurement are described by the present applicant in International Patent Publication No. WO2012/155183, the content of which is incorporated herein by reference.

Existing closed-loop systems detect a single kind of evoked neural response from neural tissue and use this to control one or more stimulus parameters. However, many instances occur where neural stimulation affects different kinds of neural tissue, e.g. different fibre types, that produce separate evoked neural responses, or a single neural tissue that produces different kinds of neural response at different stimulation intensities or with different latencies. A closed-loop system with a single neural response detector is only capable of detecting a single kind of response of a single kind of neural tissue and is therefore potentially missing out on valuable information about the efficacy of the therapy.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are closed-loop neural stimulation systems, devices, and methods that are configured to detect multiple evoked responses, and adjust the parameters of one or more stimsets based on the multiple evoked responses. The multiple responses may be evoked by the same stimulus or by different stimuli, and may be sensed via a single MEC at staggered times or via multiple MECs. The multiple responses may be combined into a single feedback variable, or used to derive multiple feedback variables. The adjustment may be to a single stimulus parameter or to multiple stimulus parameters of a single stimset, or to respective parameters of multiple stimsets in a multi-stimset CLNS system.

According to a first aspect of the present technology, there is provided an implantable device for controllably delivering neural stimuli, the device comprising: a plurality of electrodes including one or more stimulus electrodes and one or more measurement electrodes; a stimulus source configured to provide neural stimuli to be delivered via the one or more stimulus electrodes of the one or more stimulus electrodes to a neural pathway of a patient in order to evoke a neural response from the neural pathway; measurement circuitry configured to capture signal windows sensed on the neural pathway subsequent to respective neural stimuli, each signal window sensed via one or more measurement electrodes of the one or more measurement electrodes; and a control unit configured to: control the stimulus source to provide a neural stimulus according to a plurality of stimulus parameters; measure a first characteristic of a first evoked neural response in a first captured signal window subsequent to the neural stimulus; measure a second characteristic of a second evoked neural response in a second captured signal window subsequent to the neural stimulus; analyse the first measured characteristic and the second measured characteristic to determine one or more feedback variables; and adjust, using a feedback controller, the plurality of stimulus parameters so as to maintain the one or more feedback variables at respective target values.

According to a second aspect of the present technology, there is provided an automated method of controllably delivering neural stimuli, the method comprising: controlling a stimulus source to deliver a neural stimulus according to a plurality of stimulus parameters to a neural pathway of a patient in order to evoke a neural response from the neural pathway; capturing one or more signal windows sensed on the neural pathway subsequent to the delivered neural stimulus; measuring a first characteristic of a first evoked neural response in a first captured signal window of the one or more signal windows; measuring a second characteristic of a second evoked neural response in a second captured signal window of the one or more signal windows; analysing the first measured characteristic and the second measured characteristic to determine one or more feedback variables; and adjusting the plurality of stimulus parameters so as to maintain the one or more feedback variables at respective target values.

Advantages of the first and second aspect may include the ability to adjust multiple stimulus parameters enables greater selectivity of fibre type being recruited. Also, taking more neural response characteristics into account allows responses of multiple fibre types to be targeted in aggregate, such that a decrease in recruitment of one type of fibre may be balanced by an increase in recruitment of another type. Alternatively, one fibre type may be selected for recruitment at the expense of other types.

According to a third aspect of the present technology, there is provided an implantable device for controllably delivering neural stimuli, the device comprising: a plurality of electrodes including one or more stimulus electrodes and one or more measurement electrodes; a stimulus source configured to provide neural stimuli to be delivered via one or more stimulus electrodes of the one or more stimulus electrodes to a neural pathway of a patient in order to evoke a neural response from the neural pathway; measurement circuitry configured to capture signal windows sensed on the neural pathway subsequent to respective neural stimuli, each signal window sensed via one or more measurement electrodes of the one or more measurement electrodes; and a control unit configured to: control the stimulus source to provide a neural stimulus according to one or more stimulus parameters; measure a first characteristic of a first evoked neural response in a first captured signal window subsequent to the neural stimulus; measure a second characteristic of a second evoked neural response in a second captured signal window subsequent to the neural stimulus; analyse the first measured characteristic to determine a feedback variable; and adjust, using a feedback controller, one stimulus parameter of the one or more stimulus parameters based on the feedback variable and on one or more feedback loop parameters, and determine a feedback loop parameter of the one or more feedback loop parameters, or disable the feedback controller, based on the second measured characteristic.

According to a fourth aspect of the present technology, there is provided an automated method of controllably delivering neural stimuli, the method comprising: controlling a stimulus source to deliver a neural stimulus according to one or more stimulus parameters to a neural pathway of a patient in order to evoke a neural response from the neural pathway; capturing one or more signal windows sensed on the neural pathway subsequent to the delivered neural stimulus; measuring a first characteristic of a first evoked neural response in a first captured signal window of the one or more signal windows; measuring a second characteristic of a second evoked neural response in a second captured signal window of the one or more signal windows; analysing the first measured characteristic to determining a feedback variable; adjusting one stimulus parameter of the one or more stimulus parameters based on the feedback variable and on one or more feedback loop parameters; and determining a feedback loop parameter of the one or more feedback loop parameters, or disabling the adjusting, based on the second measured characteristic.

Advantages of the third and fourth aspect may include that the feedback loop is configured to maintain neural recruitment at a therapeutic target level through postural variations, while minimising any side effects.

According to a fifth aspect of the present technology, there is provided an implantable device for controllably delivering neural stimuli, the device comprising: a plurality of electrodes including one or more stimulus electrodes and one or more measurement electrodes; a stimulus source configured to provide neural stimuli to be delivered according to one of a plurality of stimulation sets to a neural pathway of a patient in order to evoke neural responses from the neural pathway, wherein each stimulation set comprises one or more stimulus electrodes of the one or more stimulus electrodes; measurement circuitry configured to capture signal windows sensed on the neural pathway subsequent to respective neural stimuli, each signal window sensed via one or more measurement electrodes of the one or more measurement electrodes; and a control unit configured to: control the stimulus source to provide a first neural stimulus according to a first stimulation set of the plurality of stimulation sets and according to one or more first stimulus parameters; control the stimulus source to provide a second neural stimulus according to a second stimulation set of the plurality of stimulation sets and according to one or more second stimulus parameters; measure a first characteristic of a first evoked neural response in a first captured signal window subsequent to the first neural stimulus; measure a second characteristic of a second evoked neural response in a second captured signal window subsequent to the second neural stimulus; adjust, using a feedback controller: the one or more first stimulus parameters so as to maintain the first measured characteristic at a first target value, and the one or more second stimulus parameters so as to maintain the second measured characteristic at a second target value; and adjust the second target value based on the first measured characteristic.

According to a sixth aspect of the present technology, there is provided an automated method of controllably delivering neural stimuli, the method comprising: controlling a stimulus source to deliver a first neural stimulus via a first stimulation set of a plurality of stimulation sets according to one or more first stimulus parameters to a neural pathway of a patient in order to evoke a first neural response from the neural pathway; controlling the stimulus source to deliver a second neural stimulus via a second stimulation set of the plurality of stimulation sets according to one or more second stimulus parameters to a neural pathway of a patient in order to evoke a second neural response from the neural pathway; capturing a plurality of signal windows sensed on the neural pathway subsequent to the delivered neural stimuli; measuring a first characteristic of the first evoked neural response in a first captured signal window of the plurality of signal windows subsequent to the first neural stimulus; measuring a second characteristic of the second evoked neural response in a second captured signal window of the plurality of signal windows subsequent to the second neural stimulus; adjusting the one or more first stimulus parameters so as to maintain the first measured characteristic at a first target value; and adjusting the one or more second stimulus parameters so as to maintain the second measured characteristic at a second target value; and adjusting the second target value based on the first measured characteristic.

Advantages of the fifth and sixth aspect may include allowing for the targeting of different fibre types by different stimsets, where their recruitments of each type may overlap to some extent.

According to a seventh aspect of the present technology, there is provided an implantable device for controllably delivering neural stimuli, the device comprising: a plurality of electrodes including one or more stimulus electrodes and one or more measurement electrodes; a stimulus source configured to provide neural stimuli to be delivered via one of a plurality of stimulation sets to a neural pathway of a patient in order to evoke neural responses from the neural pathway, wherein each stimulation set comprises one or more stimulus electrodes of the one or more stimulus electrodes; measurement circuitry configured to capture signal windows sensed on the neural pathway subsequent to respective neural stimuli, each signal window sensed via one or more measurement electrodes of the one or more measurement electrodes; and a control unit configured to: control the stimulus source to provide a first neural stimulus according to a first stimulation set of the plurality of stimulation sets and according to one or more first stimulus parameters; control the stimulus source to provide a second neural stimulus according to a second stimulation set of the plurality of stimulation sets and according to one or more second stimulus parameters; measure a first characteristic of a first evoked neural response in a first captured signal window subsequent to the first neural stimulus; measure a second characteristic of a second evoked neural response in a second captured signal window subsequent to the second neural stimulus; and adjust, using a feedback controller: the one or more first stimulus parameters so as to maintain the first measured characteristic at a first target value, a second stimulus parameter of the one or more second stimulus parameters so as to maintain the second measured characteristic at a second target value, and adjust a further second stimulus parameter of the one or more second stimulus parameters based on the adjustment to the one or more first stimulus parameters.

According to an eighth aspect of the present technology, there is provided an automated method of controllably delivering neural stimuli, the method comprising: controlling a stimulus source to deliver a first neural stimulus via a first stimulation set of a plurality of stimulation sets according to one or more first stimulus parameters to a neural pathway of a patient in order to evoke a first neural response from the neural pathway; controlling the stimulus source to deliver a second neural stimulus via a second stimulation set of the plurality of stimulation sets according to one or more second stimulus parameters to a neural pathway of a patient in order to evoke a second neural response from the neural pathway; capturing a plurality of signal windows sensed on the neural pathway subsequent to the delivered neural stimuli; measuring a first characteristic of the first evoked neural response in a first captured signal window of the plurality of signal windows subsequent to the first neural stimulus; measuring a second characteristic of the second evoked neural response in a second captured signal window of the plurality of signal windows subsequent to the second neural stimulus; adjusting the one or more first stimulus parameters so as to maintain the first measured characteristic at a first target value; adjusting a first stimulus parameter of the one or more second stimulus parameters so as to maintain the second measured characteristic at a second target value; and adjusting a further second stimulus parameter of the one or more second stimulus parameters based on the adjustment to the one or more first stimulus parameters.

Advantages of the seventh and eighth aspect may include allowing multiple sub-stimsets that are adjusted ratiometrically to remain sub-paresthesia-threshold.

According to a ninth aspect of the present technology, there is provided an implantable device for controllably delivering neural stimuli, the device comprising: a plurality of electrodes including one or more stimulus electrodes and one or more measurement electrodes; a stimulus source configured to provide neural stimuli to be delivered via the one or more stimulus electrodes of the one or more stimulus electrodes to a neural pathway of a patient in order to evoke a neural response from the neural pathway; measurement circuitry configured to capture signal windows sensed on the neural pathway subsequent to respective neural stimuli, each signal window sensed via one or more measurement electrodes of the one or more measurement electrodes; and a control unit configured to: control the stimulus source to provide a neural stimulus according to one or more stimulus parameters; measure a first characteristic of a first evoked neural response in a first captured signal window subsequent to the neural stimulus; measure a second characteristic of a second evoked neural response in the first captured signal window subsequent to the neural stimulus; analyse the first measured characteristic to determine a first feedback variable; analyse the second measured characteristic to determine a second feedback variable; and adjust, using a feedback controller, one stimulus parameter of the one or more stimulus parameters so as to maintain an active feedback variable of the first and second feedback variables at an active target value of a first target value and a second target value.

According to a tenth aspect of the present technology, there is provided an automated method of controllably delivering neural stimuli, the method comprising: controlling a stimulus source to deliver a neural stimulus according to one or more stimulus parameters to a neural pathway of a patient in order to evoke a neural response from the neural pathway; capturing one or more signal windows sensed on the neural pathway subsequent to the delivered neural stimulus; measuring a first characteristic of a first evoked neural response in a first captured signal window of the one or more signal windows; measuring a second characteristic of a second evoked neural response in the first captured signal window; analysing the first measured characteristic to determine a first feedback variable; analysing the second measured characteristic to determine a second feedback variable; and adjusting one stimulus parameter of the one or more stimulus parameters so as to maintain an active feedback variable of the first and second feedback variables at an active target value of a first target value and a second target value.

Advantages of the ninth and tenth aspect may include loop responsivity for a feedback loop on one characteristic in the saturation region of that characteristic by driving the loop using another, non-saturating characteristic.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is an illustration of the stimulus pulses delivered by a stimulation program with four interleaved stimulation sets (stimsets);

FIG. 9 is a schematic illustrating elements and inputs of a closed-loop neural stimulation (CLNS) system with multiple stimsets;

FIG. 10 is a schematic illustrating elements of a multi-response single-stimset CLNS system, according to one aspect of the present technology;

FIG. 11 is a flowchart illustrating a method of operating a multi-response, single-stimset feedback loop according to one aspect of the present technology;

FIG. 12 contains a graph containing growth curves for two different measured characteristics;

FIG. 13 is a schematic illustrating elements of a multi-response multi-stimset CLNS system, according to a second aspect of the present technology; and

FIG. 14 is a flowchart illustrating a method of operating a multi-response, multi-stimset feedback loop according to the second aspect of the present technology.

### DETAILED DESCRIPTION OF THE PRESENT TECHNOLOGY

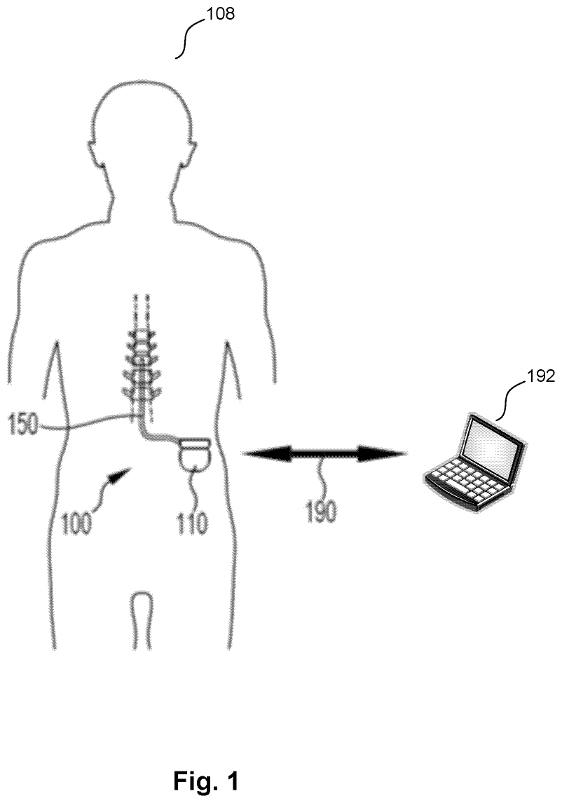

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

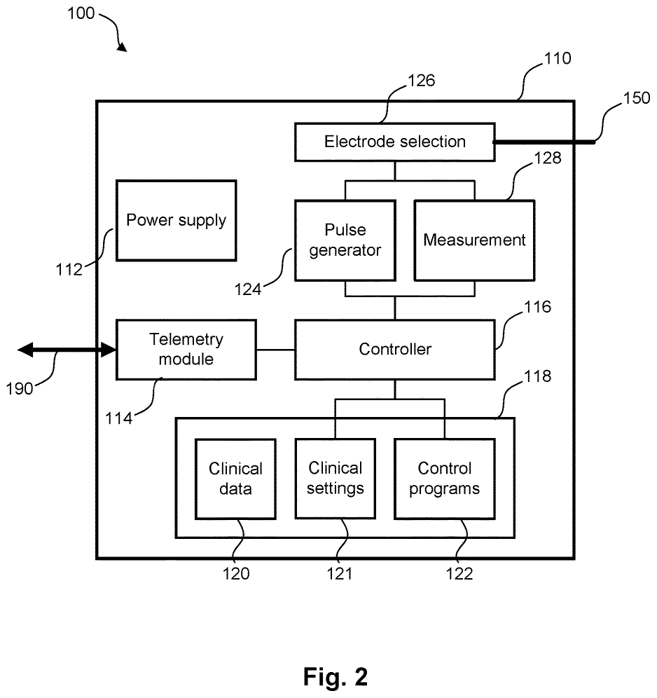

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

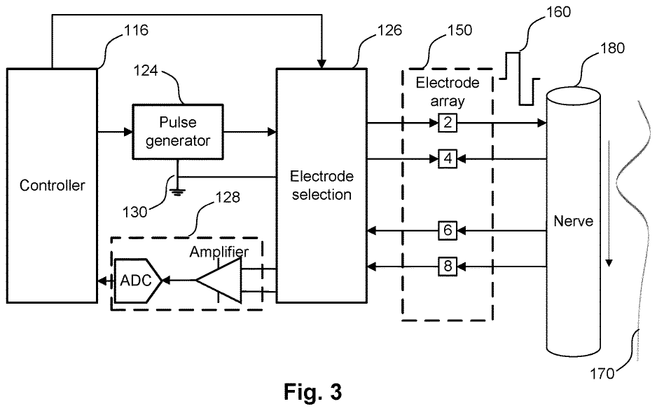

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative embodiments may apply other forms of bipolar stimulation, or may use a greater number of stimulus and/or return electrodes. The set of stimulus and return electrodes and their respective polarities is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via stimulus electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain and of a quality that is comfortable for the patient, the clinician or the patient nominates that configuration for ongoing use. The program parameters may be loaded into the memory 118 of the stimulator 100 as the clinical settings 121.

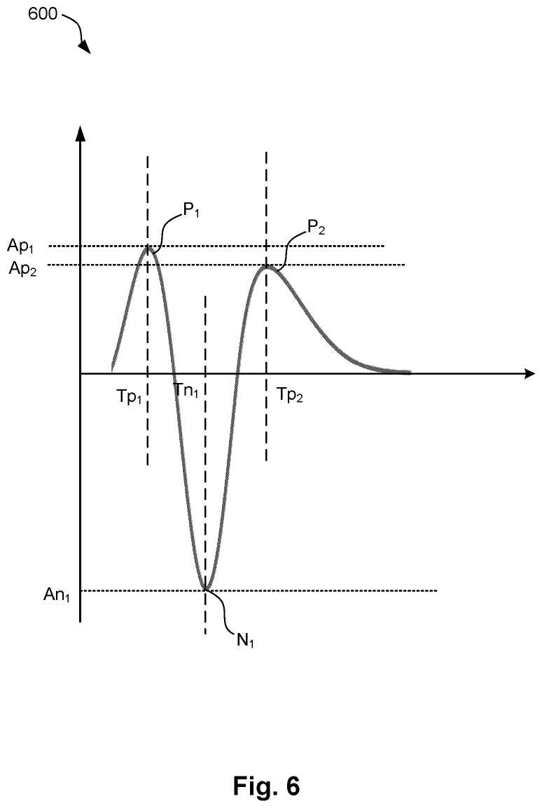

FIG. 6 illustrates the typical form of an ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The time of occurrence of a peak may be referred to as the latency of a peak. The peak-to-peak amplitude is Ap₁+An₁. A recorded ECAP will typically have a maximum peak-to-peak amplitude in the range of microvolts and a duration of 2 to 3 ms.

The stimulator 100 is further configured to detect the existence and measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may extract and store two or more characteristics from the neural response.

Stimulator 100 applies stimuli over a potentially long period such as days, weeks, or months and during this time may store characteristics of neural responses, clinical settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

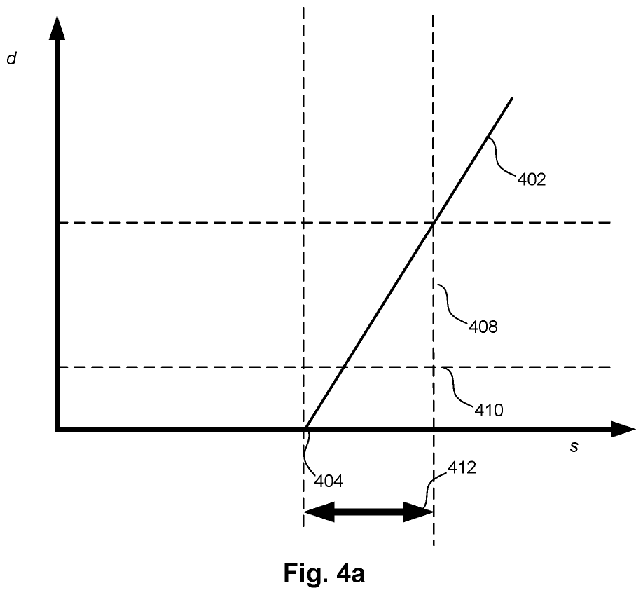

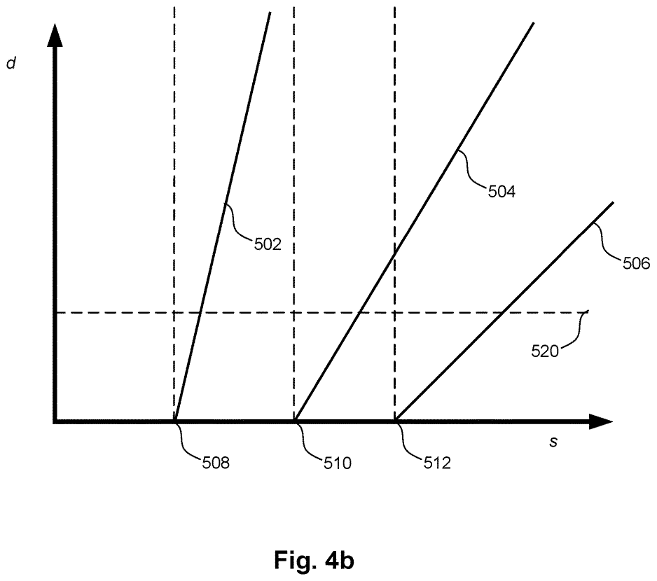

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus intensity, y is the ECAP amplitude, Tis the ECAP threshold and Sis the slope of the activation plot (referred to herein as the patient sensitivity). The slope S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulation (CLNS) device. By adjusting the applied stimulus intensity to maintain the measured ECAP amplitude at an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple stimulus parameters including stimulus pulse amplitude, pulse width, number of phases, order of phases, number of stimulus electrode poles (two for bipolar, three for tripolar etc.), and stimulus rate or frequency. At least one of the stimulus parameters, for example the stimulus intensity, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is detected, and its amplitude measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

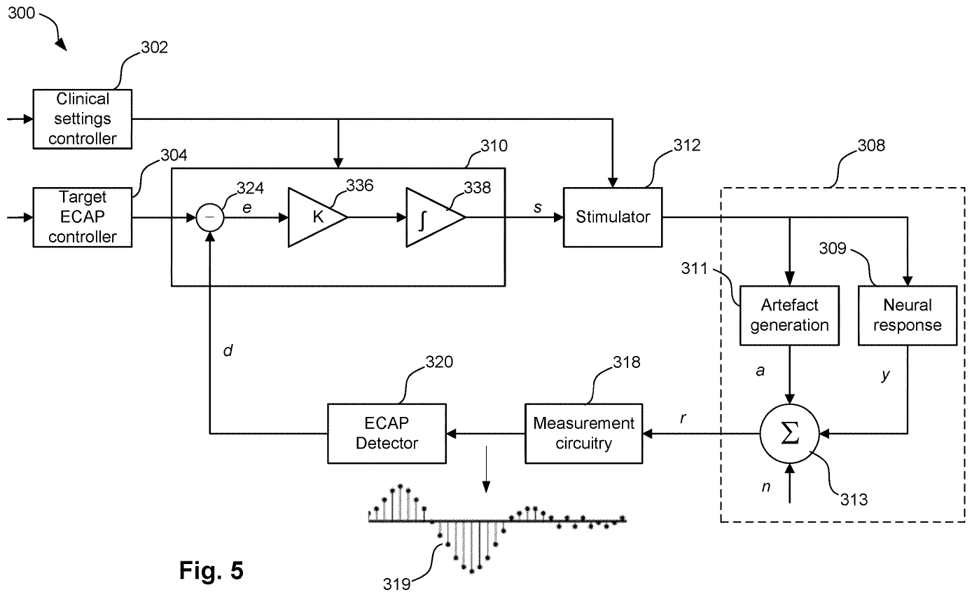

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in company with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the neural tissue of the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and measurement noise) and samples the amplified sensed signal r to capture a “signal window” 319 comprising a predetermined number of samples of the amplified sensed signal r. The ECAP detector 320 processes the signal window 319 and outputs a measured neural response intensity d. In one implementation, the neural response intensity comprises a peak-to-peak ECAP amplitude. The measured response intensity d is input into the feedback controller 310. The feedback controller 310 comprises a comparator 324 that compares the measured response intensity d (an example of a feedback variable) to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

$$s = \int K e d t \qquad ( 2 )$$

where K is the gain of the gain element 336 (the controller gain). This relation may also be represented as

$$δ s = K e \qquad ( 3 )$$

where δₛ is an adjustment to the current stimulus intensity parameter s.

A target ECAP amplitude is input to the feedback controller 310 via the target ECAP controller 304. In one embodiment, the target ECAP controller 304 provides an indication of a specific target ECAP amplitude. In another embodiment, the target ECAP controller 304 provides an indication to increase or to decrease the present target ECAP amplitude. The target ECAP controller 304 may comprise an input into the CLNS system 300, via which the patient or clinician can input or adjust a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

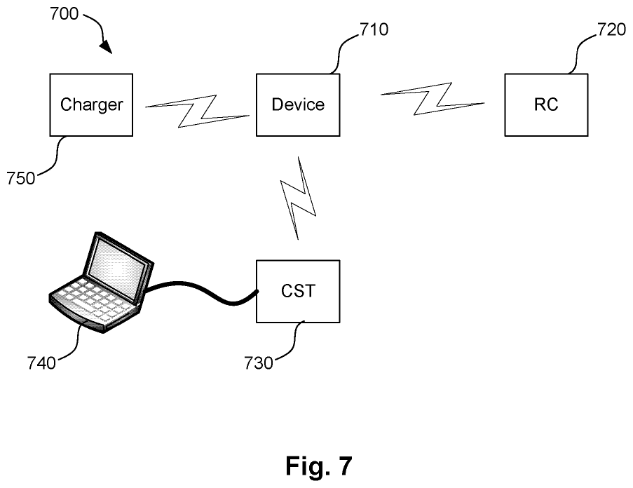

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### Late Response

International Patent Publication no. WO2015070281, the contents of which are herein incorporated by reference, describes the slow or late response to neural stimulation. When a stimulus is applied to neural tissue, under certain circumstances a late response (LR) is discernible in the sensed signal following the ECAP, and carries important information. The late responses, unlike ECAPs, are not the direct response of the neural tissue but rather appear to be a systems response from the cortex and other subcortical structures projecting back into the sub-thalamic nucleus of the brain. Late responses are typically of much smaller amplitude than the ECAP, and typically do not have a linear growth curve. Late responses undergo three distinct states when the stimulation intensity is increased: the state when the stimulation intensity is below the late response threshold, where no late response is evoked; the non-therapeutic state in which a clear late response is present but which has no therapeutic effect for the patient; and the therapeutic state, which coincides with the neurologist's assessment of therapeutic levels of stimulation intensities.

### Multi-Stimset Neural Stimulation

For some patients, it is beneficial for a neural stimulation therapy program to comprise multiple stimulation sets. A stimulation set (“stimset”) is a set of stimulus and return electrodes, or more precisely a stimulus electrode configuration (SEC), along with the stimulus parameters that govern the stimulation pulses delivered via that SEC.

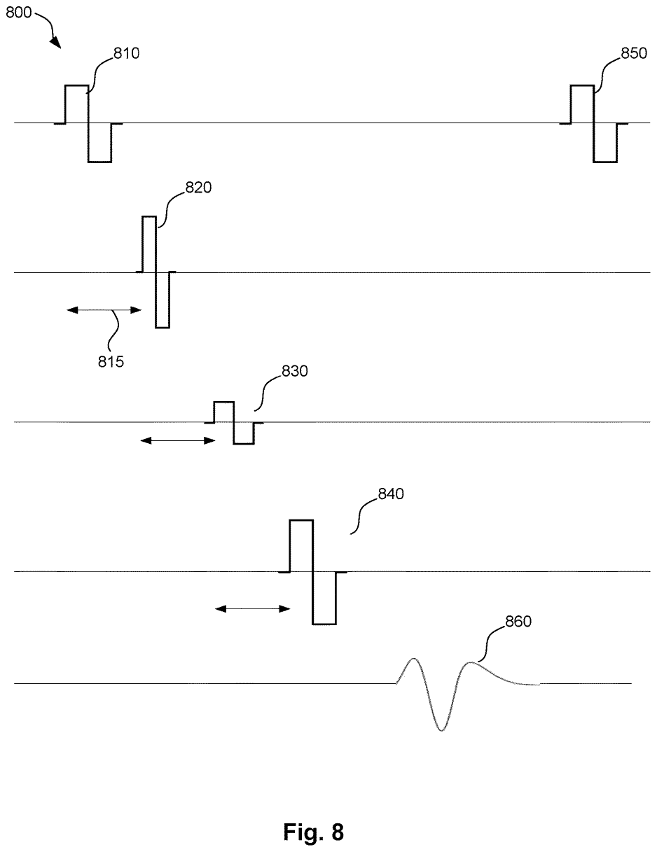

FIG. 8 is an illustration 800 of the stimulus pulses delivered by a stimulation program with four interleaved stimsets. The stimulus pulse train delivered according to each stimset is illustrated on a separate, but vertically aligned, horizontal axis representing time. All the stimulus pulse trains are delivered at the same stimulus frequency. (It is not a requirement that all the stimulus pulse trains for the respective stimsets are delivered at the same stimulus frequency; however it is so represented in FIG. 8 for ease of illustration.) The first stimulus pulse 810, delivered according to the first stimset, is illustrated as a biphasic, anodic-first stimulus pulse, though many other stimulus pulse types are contemplated. The second, third, and fourth stimulus pulses 820, 830, and 840, delivered according to the second, third, and fourth stimsets in the program respectively, are also biphasic, anodic-first stimulus pulses with different pulse widths and different amplitudes. Each stimulus pulse is illustrated as delayed in time by a constant amount (the inter-stimulus interval, or ISI, 815) from the stimulus pulse delivered according to the preceding stimset. However, this is not to be interpreted as limiting, since the intervals between the pulses in the various stimsets may be different. Because all the stimulus pulse trains in FIG. 8 are delivered at the same stimulus frequency, the four stimulus pulses 810, 820, 830, 840 form a cycle that repeats indefinitely without any change to the relative timing of the pulses from the different stimsets. The fifth stimulus pulse 850 is a subsequent pulse in the pulse train delivered according to the first stimset and is therefore illustrated on the same time axis as the first stimulus pulse 810, and the cycle repeats thereafter.

Also illustrated is an evoked neural response in the form of an evoked compound action potential (ECAP) 860 as sensed via a predetermined measurement electrode configuration (MEC) on a common time axis with the stimulus pulses. The illustrated ECAP 860 is evoked by the fourth stimulus pulse 840. A closed-loop neural stimulation (CLNS) system programmed with multiple interleaved stimsets, as illustrated in FIG. 8, may be based on measurements of the ECAP 860. That is to say, closed-loop adjustments to the stimulus parameters of all stimsets may all be based on measurements of the ECAP 860 from a single stimset, referred to as the applied stimset. In FIG. 8, the final stimset in the cycle is the applied stimset.

If the ISI 815 is short, ECAPs evoked by the first three stimulus pulses 810, 820, and 830 are potentially obscured by stimulus crosstalk and/or artefact from the stimulus pulses 820, 830, and 840. Therefore, if the ISI 815 is short, only the final stimset in the cycle may evoke a measurable ECAP. If the ISI 815 is greater than the refractory period and sufficiently long that ECAPs evoked by the earlier stimsets are not obscured by stimulus crosstalk and artefact from the other stimulus pulses in the cycle, any of the stimsets in the cycle may evoke a measurable ECAP.

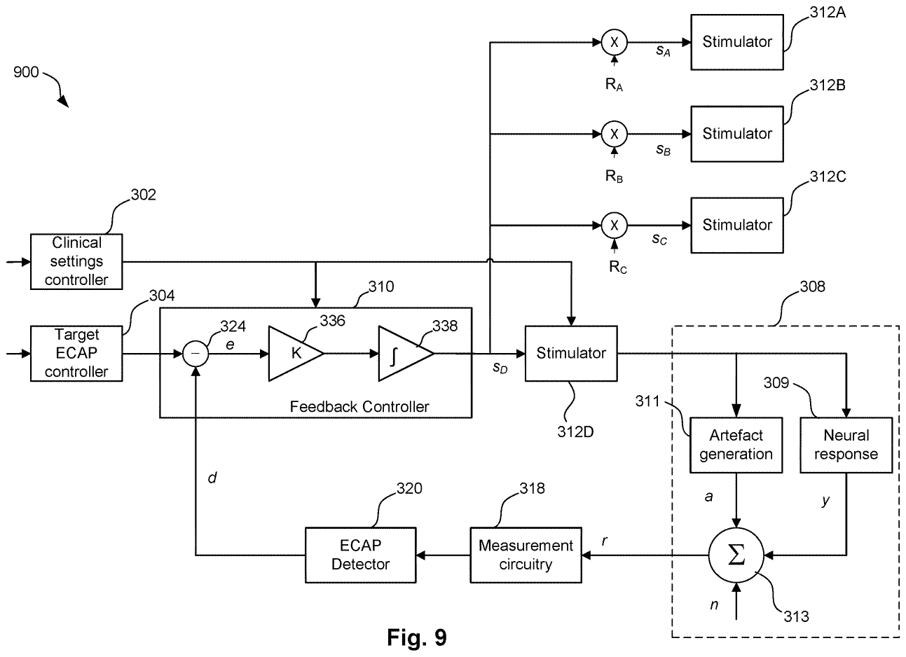

FIG. 9 is a schematic illustrating elements and inputs of a multi-stimset CLNS system 900 with multiple stimsets. The multi-stimset CLNS system 900 is the same as the CLNS system 300 of FIG. 5, with like numbers indicating like elements, with the addition of three further stimsets. The four stimsets are labelled A, B, C, and D and are delivered by stimulators 312A, 312B, 312C, and 312D (the latter of which corresponds to the stimulator 312 in the CLNS system 300) according to respective stimulus intensity parameters S_{A}, S_{B}, S_{C}, and S_{D}, and via respective SECs. The pulses delivered by the stimulators 312A, 312B, 312C, and 312D correspond to the stimulus pulses 810, 820, 830, and 840 of FIG. 8. Stimset D, delivered by the stimulator 312D, is delivered last in the cycle and is the applied stimset, from which the ECAP is measured. In the implementation of FIG. 8, the stimulus intensity parameter S_{D} for stimset D is scaled by ratios R_{A}, R_{B}, and R_{C} to obtain the stimulus intensity parameters S_{A}, S_{B}, and S_{C} for stimsets A, B, and C respectively. The ratios R_{A}, R_{B}, and R_{C} are fixed at the ratios of the respective stimulus intensities at which the respective stimsets were originally programmed, to the originally programmed stimulus intensity of the applied stimset D. In such an implementation, the stimulus intensity parameters S_{A}, S_{B}, and S_{C} always remain in fixed ratio with the applied stimulus intensity parameter S_{D} and with each other. This is referred to as ratiometric adjustment. So for example, if the originally programmed stimulus intensities were 1 mA, 2 mA, 4 mA, and 6 mA for the four stimsets A, B, C, and D respectively, the ratios R_{A}, R_{B}, and R_{C} are fixed at programming time at ⅙, ⅓, and ⅔ respectively and form part of the clinical settings 121 of the multi-stimset program. If during therapy the feedback controller 310 adjusts the applied stimset intensity parameter S_{D} to 6.6 mA, the stimulus intensity parameters S_{A}, S_{B}, and S_{C} of the non-applied stimsets are automatically adjusted to 1.1 mA, 2.2 mA, and 4.4 mA respectively. The clinical settings controller 302 provides to the stimulators 312A, 312B, 312C, and 312D the stimulus parameters that are not under the control of the feedback controller 310.

It may be seen from FIG. 9 that the adjustments to the stimulus intensity parameters after each stimulus cycle are all in fixed proportion. A ratiometric multi-stimset CLNS system therefore emulates a CLNS system with four separate feedback loops driven by the four stimsets, wherein each loop has the same controller gain. A ratiometric multi-stimset CLNS system is effective to maintain the responses evoked by each stimset at a constant neural response intensity on the condition that when the patient moves to a new posture, the threshold and slope of all activation plots, both for applied and non-applied stimsets, move in a proportional manner. (See FIG. 4b for examples of activation plots for a given stimset in different postures.)

### Multiple Response Feedback Control

Existing CLNS systems such as the CLNS system 300 or the ratiometric multi-stimset CLNS system 900 measure a single characteristic of evoked neural responses from neural tissue and use this to control one or more stimulus parameters. However, as mentioned above, many instances occur where neural stimulation affects different kinds of neural tissue, e.g. different fibre types, that produce separate evoked neural responses, or a single kind of neural tissue that produces different kinds of neural response, e.g. ECAPs and late responses, at different stimulation intensities. A CLNS system with a single neural response detector is only capable of detecting a single kind of response of a single kind of neural tissue and is therefore potentially missing out on valuable information about the efficacy of the therapy.

As one example, suppose one wished to operate a feedback loop stimulating the dorsal column just above the threshold of the late response. This would be difficult, as stimuli below the late response threshold do not evoke late responses, and so for a significant proportion of the time there would be no meaningful error signal to use to adjust the stimulation intensity.

CLNS systems, devices, and methods according to the present technology are configured to measure multiple characteristics of evoked neural responses, and adjust the parameters of one or more stimsets based on the multiple measured characteristics. The multiple characteristics may be evoked by the same stimulus or by different stimuli, and may be sensed via a single MEC at staggered times or via multiple respective MECs. The multiple characteristics may be combined into a single feedback variable, or used to derive multiple feedback variables. The adjustment may be to a single stimulus parameter or to multiple stimulus parameters of a single stimset, or to respective parameters of multiple stimsets in a multi-stimset CLNS system.

According to a first aspect of the present technology, a single-stimset CNLS system is configured to measure characteristics of multiple types of evoked neural response to neural stimuli. The multiple evoked neural response characteristics are used to control one or more parameters of the delivered neural stimuli.

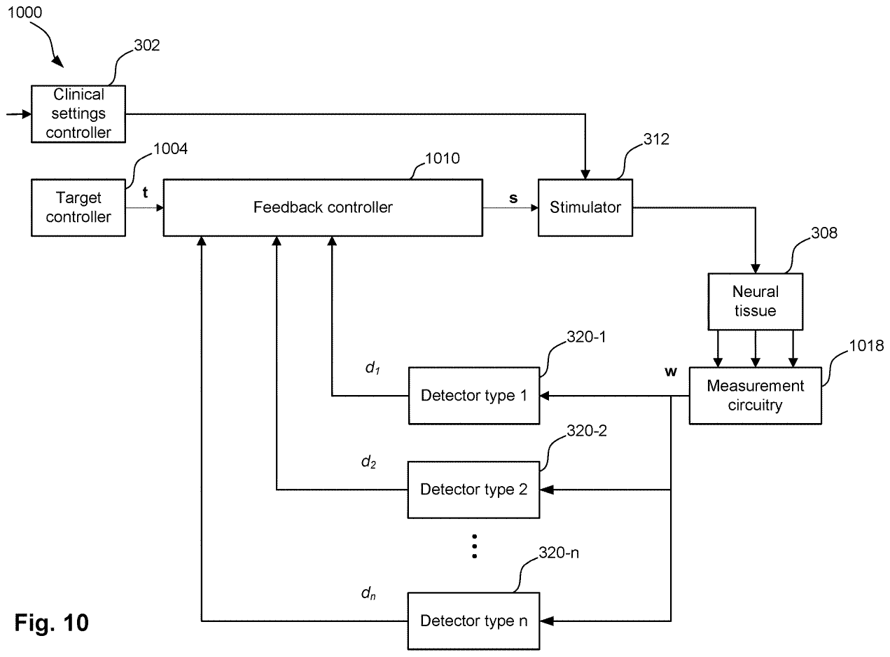

FIG. 10 is a schematic illustrating elements of a multi-response single-stimset CLNS system 1000, according to the first aspect of the present technology. The multi-response single-stimset CLNS system 1000 is the same as the CLNS system 300 of FIG. 5, with like numbers indicating like elements, except that certain elements have been simplified, others have been replaced entirely, and new elements have been added. In particular, the internal contents of the box 308 have been removed for simplicity. The measurement circuitry 1018, which replaces measurement circuitry 318 and which receives input from one or more MECs (three are illustrated in FIG. 10), generates a window vector w comprising one or more components. Each component of the window vector w represents a signal window of samples of an amplified signal sensed via a corresponding MEC at a predetermined time subsequent to a delivered neural stimulus. The single ECAP detector 320 has been replaced by multiple response detectors 320-1, 320-2, . . . , 320-n (n>1), each of which analyses a corresponding signal window of the window vector w. Multiple response detectors 320-i may analyse the same signal window of the window vector w. Each response detector 320-i returns a corresponding characteristic dᵢ of the signal window. The characteristic dᵢ may be a scalar-valued measurement of a characteristic of a corresponding kind of neural response, or a Boolean value indicating the presence or absence of its corresponding kind of neural response. For example, a response detector 320-i may measure the amplitude of an ECAP from a particular fibre type in a signal window of the window vector w. A different response detector 320-j may detect the presence of a late response from the same fibre type in the same signal window, or a different signal window, of the window vector w.

In some implementations, the signal windows making up the window vector w may be signal windows of the sensed signal r captured via the same MEC at different times subsequent to a neural stimulus. In some such implementations, the signal windows may all be captured subsequent to the same neural stimulus. Alternatively, in other such implementations, at least two of the signal windows may be captured subsequent to different neural stimuli. In this latter case, at least two neural stimuli need to be delivered before all the signal windows are available to make up the window vector w.

In other implementations, the signal windows making up the window vector w may be signal windows of the sensed signal r captured via different MECs subsequent to a single neural stimulus. In some such implementations, the signal windows may all be captured simultaneously subsequent to the same neural stimulus. In other such implementations, at least two of the signal windows may be captured at different times subsequent to the same neural stimulus.

The single-response, single-parameter feedback controller 310 and all its constituent elements have been replaced by a multi-response feedback controller 1010. The multi-response feedback controller 1010 receives and analyses the characteristics d₁, d₂, . . . , dₙ from the respective response detectors 320-1, 320-2, . . . , 320-n to generate a stimulus parameter vector s. The stimulus parameter vector s comprises one or more stimulus parameters that govern the delivery of each stimulus by the stimulator 312. The target ECAP controller 304 has been replaced by a more general target controller 1004 that feeds a target vector t to the multi-response feedback controller 1010.

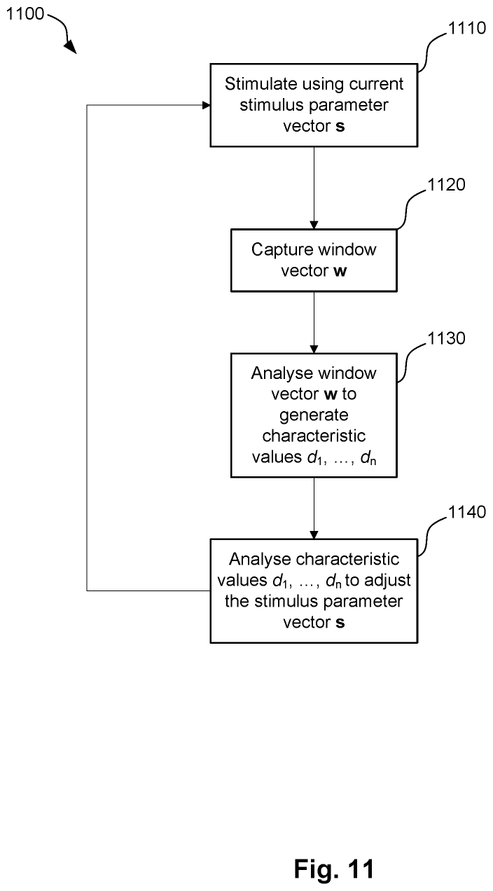

FIG. 11 is a flowchart illustrating a method 1100 of operating a multi-response, single-stimset feedback loop according to one aspect of the present technology. The method 1100 may be implemented by the multi-response single-stimset CLNS system 1000 of FIG. 10. The method 1100 starts at step 1110, at which the stimulator 312 delivers a neural stimulus in accordance with the current value of the stimulus parameter vector s via the SEC of the single stimset. Step 1120 then captures the window vector w subsequent to the delivered stimulus from step 1110. (In some implementations using a single MEC, as described above, the method 1100 may need to loop back from step 1120 to step 1110 at least once to deliver more stimuli until the window vector w is fully populated with signal windows.) Step 1130 uses the neural response detectors 320-1, 320-2, . . . , 320-n to generate the characteristics d₁, d₂, . . . , dₙ from the window vector w. At step 1140, the multi-response feedback controller 1010 analyses the characteristics d₁, d₂, . . . , dₙ from the neural response detectors 320-1, 320-2, . . . , 320-n and the target vector t from the target controller 1004 to adjust the stimulus parameter vector s. The method 1100 then loops back to step 1110 to deliver the next neural stimulus using the adjusted stimulus parameter vector s.

According to a first implementation of the multi-response single-stimset CLNS system 1000 and the method 1100, the window vector w captured at step 1120 comprises two component signal windows w₁ and w₂. To implement step 1130, the response detector 320-1 measures a first characteristic d₁ of an ECAP in the signal window w₁, and the response detector 320-2 measures a second characteristic d₂ of the ECAP in the signal window w₂. The target vector t comprises a target value t₁ for the first characteristic and a target value t₂ for the second characteristic. To implement step 1140, the multi-response feedback controller 1010 adjusts a first stimulus parameter s₁ after each delivered stimulus to maintain the first characteristic at the target value t₁ for the first characteristic, and independently adjusts a second stimulus parameter s₂ after each delivered stimulus to maintain the second characteristic at the target value t₂ for the second characteristic. This may be extended to three or more characteristics, each with a corresponding target value and a corresponding stimulus parameter. The multi-response feedback controller 1010 according to this first implementation may be implemented using the same number of independent instances of the single-response, single-parameter feedback controller 310 described above.

In this first implementation, there needs to be some causal link between each stimulus parameter and its corresponding ECAP characteristic, so that adjusting each stimulus parameter affects its corresponding ECAP characteristic.

One example of the first implementation is:
the first ECAP characteristic is the ECAP amplitude;
the first stimulus parameter is stimulus intensity;
the second ECAP characteristic is the N1 peak latency;
the second stimulus parameter is pulse width.

In this example of the first implementation, recruitment of a particular fibre type (with a conduction velocity dependent on the target N1 peak latency) will be maintained at a target level.

In another example of the first implementation, the electrode array is positioned obliquely to the dorsal column, so at least some electrodes are proximate to the dorsal root ganglion. To implement step 1130, the response detector 320-1 measures the amplitude d₁ of an ECAP from a particular fibre type in a signal window w₁, and the response detector 320-2 measures the amplitude d₂ of a late response from the same fibre type, except from a different MEC and therefore in a different signal window w₂. The MEC at which the late response is detected is located near the dorsal root ganglion of the fibre type being targeted by the response detector 320-1. To implement step 1140, the multi-response feedback controller 1010 adjusts stimulus intensity after each delivered stimulus to maintain the ECAP amplitude d₁ at its target value t₁. The target value t₂ for the late response amplitude is set to zero, and the corresponding stimulus parameter s₂ is the central point of stimulus (CPS). The multi-response feedback controller 1010 therefore adjusts the CPS after each delivered stimulus to maintain a zero late response amplitude. Moving the CPS medially reduces the late response amplitude, so the multi-response feedback controller 1010 will maintain the CPS just laterally enough so that the late response is undetectable, while maintaining the ECAP amplitude at its target by adjusting the stimulus intensity.

In another example of the first implementation of the multi-response single-stimset CLNS system 1000 and the method 1100, to implement step 1130, the response detector 320-1 measures the amplitude d₁ of an ECAP in a signal window w₁, and the response detector 320-2 measures the principal frequency d₂ of non-evoked neural activity in a different signal window w₂ that is timed to contain no evoked neural responses. To implement step 1120, the signal window w₂ may be captured via the same MEC as the signal window w₁, except delayed in time from w₁ so that the evoked neural response has finished by the time w₂ begins. Alternatively, the signal window w₂ may be captured via a different MEC from the signal window w₁, located a site far from the stimulus site (e.g. at the dorsal root). The principal frequency of non-evoked neural activity in the signal window w₂ may be measured by finding the frequency of the largest peak of the magnitude of the Fast Fourier Transform of the signal window w₂. The target vector t comprises a target value t₁ for the measured ECAP amplitude d₁. To implement step 1140, the multi-response feedback controller 1010 adjusts stimulus intensity after each delivered stimulus to maintain the measured ECAP amplitude d₁ at its target value t₁. The multi-response feedback controller 1010 then adjusts the stimulus frequency to be equal to the measured principal frequency d₂ of non-evoked neural activity.

A variation of the first implementation is for the multi-response feedback controller 1010 to adjust, at step 1140, each stimulus parameter s after each delivered stimulus based on all the measured characteristics d₁ to jointly maintain each measured characteristic d₁ as close as possible to its corresponding target value t₁. The multi-response feedback controller 1010 in this variation may be implemented as a Kalman filter.

According to a second implementation of the multi-response single-stimset CLNS system 1000 and the method 1100, to implement step 1140, the multi-response feedback controller 1010 combines the measured characteristics d₁, d₂, . . . , dₙ into a single, combined feedback variable d. The multi-response feedback controller 1010 then adjusts a single stimulus parameter s after each delivered stimulus to maintain the combined feedback variable d at a single target value t provided by the target controller 1004.

In one example of the second implementation, each response detector 320-i measures the amplitude d₁ of an ECAP from a particular fibre type in a signal window of the window vector w. The combined feedback variable d is determined at step 1140 as a weighted sum:

$$d = {\sum }_{i = 1}^{n} {a}_{i} {d}_{i} \qquad ( 4 )$$

where the parameters a₁, a₂, . . . , aₙ are the weights for the respective measured characteristics d₁, d₂, . . . , dₙ. This implementation allows responses of multiple fibre types to be targeted in aggregate, such that a decrease in recruitment of one type of fibre may be balanced by an increase in recruitment of another type. The weights a₁, a₂, . . . , aₙ may be chosen to favour a particular fibre type over the others, by making the weight corresponding to that fibre type larger than the other weights. The multi-response single-stimset CLNS system will therefore adjust a stimulus parameter to evoke responses of the target amplitude from the favoured fibre type.

Another example of the second implementation is configured to address flexion (bending) of the electrode array caused by posture change. Flexion of the electrode array causes electrode-to-cord distance to change differentially along the array. In other words, flexion causes the distance between some electrodes and the spinal cord to change more than the distance between other electrodes and the spinal cord. According to this example, the window vector w captured at step 1120 comprises two component signal windows w₁ and w₂, measured using MECs positioned at opposite ends of the array. To implement step 1130, the response detector 320-1 measures a first characteristic d₁ of an ECAP in the first signal window w₁, and the response detector 320-2 measures a second characteristic d₂ of the ECAP in the signal window w₂. To implement step 1140, the combined feedback variable d is determined as a weighted sum as in Equation (4). The weights a₁ and az are set equal to one another so that the combined feedback variable d is proportional to the average of d₁ and d₂. This example improves the robustness of the loop to flexion of the array.

In a third example of the second implementation, to implement step 1140, the combined feedback variable d is determined as a weighted sum as in Equation (4). However, the weighted sum contains only one term, but the weight a₁ of the measured characteristic d₁ depends on a second measured characteristic d₂ of the signal window w:

$$d = {a}_{1} ( {d}_{2} ) {d}_{1} \qquad ( 5 )$$

In an example of this example of the second implementation, the weight a₁ may be a function of the measured latency d₂ of the ECAP peak whose measured amplitude is d₁. The function is chosen so that weight a₁(d₂) is greatest when the measured latency d₂ equals the latency that would result from ECAPs propagating in fibres of a predetermined preferred conduction velocity. The effect is that recruitment of fibres of a certain diameter (i.e. those that yield ECAPs that propagate at the preferred conduction velocity) is selectively encouraged by the multi-response feedback controller 1010.

According to a third implementation of the multi-response single-stimset CLNS system 1000 and the method 1100, one or more secondary measured characteristics dⱼ (for j>1), possibly in conjunction with their respective target values tⱼ, determine a feedback loop parameter of the primary feedback loop, i.e. the feedback loop that is driven by the primary measured characteristic d₁. Examples of primary feedback loop parameters are: the target value t₁ for the primary measured characteristic d₁; the controller gain K₁ that is used to adjust the stimulus parameter s₁; or parameters of a Kalman filter implementation of a feedback controller such as state uncertainty or process noise. Alternatively, the one or more secondary measured characteristics dⱼ (for j>1), possibly in conjunction with their respective target values t₁, determine a feedback loop state of the primary feedback loop. An example of the feedback loop state is the state of the integrator 338, which may be set to zero or held unchanged.

In a first example of the third implementation, to implement step 1130, the response detector 320-1 measures the amplitude d₁ of an ECAP from a particular fibre type in the signal window w, and the response detector 320-2 detects the presence of a late response from the same fibre type in the same signal window. The characteristic d₂ is therefore a Boolean value which, when True, indicates the presence of a late response in the signal window w, rather than a scalar-valued amplitude. The target controller 1004 provides a static target ECAP amplitude T₁ to the multi-response feedback controller 1010. The controller 116 repeatedly determines a dynamic target ECAP amplitude t₁ by default at the static target ECAP amplitude T₁. To implement step 1140, the multi-response feedback controller 1010 adjusts the stimulus intensity s after each delivered stimulus to maintain the ECAP amplitude d₁ at the dynamic target ECAP amplitude t₁. The multi-response feedback controller 1010 keeps the dynamic target ECAP amplitude t₁ at the static target ECAP amplitude T₁ as long as the characteristic d₂ is False. If the characteristic d₂ is True, the multi-response feedback controller 1010 temporarily decreases the stimulus intensity until the characteristic d₂ returns to False, i.e. until a late response is no longer detected. The controller 116 may achieve this temporary decrease in stimulus intensity by, for example, temporarily decreasing the dynamic target ECAP amplitude t₁ below the static target ECAP amplitude T₁ until the characteristic d₂ returns to False.

According to this first example of the third implementation, the feedback loop is configured to maintain neural recruitment at a therapeutic target level through postural variations, while minimising any side effects of which the late response is a biomarker.

According to a second example of the third implementation of the multi-response single-stimset CLNS system 1000 and the method 1100, one or more secondary measured characteristics dⱼ (for j>1), possibly in conjunction with their respective target values tⱼ, determine a controller gain K₁ for the primary feedback loop. The controller 116 determines the controller gain K₁ for the primary feedback loop based on the one or more secondary measured characteristics dⱼ, possibly in conjunction with their respective target values tⱼ. To implement step 1140, the multi-response feedback controller 1010 adjusts a stimulus parameter s after each delivered stimulus to maintain the primary measured characteristic d₁ at its target value t₁ using the primary controller gain K₁. The multi-response feedback controller 1010 may be implemented using the single-response, single-parameter feedback controller 310 described above.

In this second example of the third implementation, the window vector w captured at step 1120 comprises a single component signal window w. To implement step 1130, the response detector 320-1 measures the amplitude d₁ of an ECAP from a particular fibre type in the signal window w, and the response detector 320-2 measures the amplitude d₂ of a late response from the same fibre type in the signal window w. The controller 116 determines the controller gain K₁ for the primary feedback loop dependent on the measured amplitude d₂ of the late response such that K₁ increases above a default value if the late response amplitude d₂ becomes significantly greater than zero. Alternatively, the response detector 320-2 may detect the presence of a late response from the same fibre type in the signal window w, so that d₂ is a Boolean value. The controller 116 internally switches the controller gain K₁ between two values, a lower, default value if d₂ is False, and a higher value if d₂ is True. The benefit of this second example of the third implementation can be felt when the patient coughs. Stimuli delivered during coughs often evoke a late response before the ECAP, particularly at lower stimulus frequencies. The resulting increase in the controller gain means that stimulus intensity is turned down to a greater degree for a given error of the ECAP amplitude from the ECAP target value than during normal operation, reducing the chances of temporary over-stimulation as a result of the cough.

A third example of the third implementation is configured to address the phenomenon of adaptation. Adaptation is the term used for a decrease in measured ECAP amplitude in response to stimulus of a fixed intensity over an initial period after turning on stimulation at a given SEC. The initial period typically lasts for a second or two. Once adaptation is complete, the ECAP amplitude levels out. During adaptation, the patient may feel no change in sensation. It is hypothesised that certain measurable characteristics of the ECAP indicate the progress of adaptation, e.g. by increasing or decreasing, then levelling out in sync with the change in ECAP amplitude during adaptation. One such characteristic may be the N1 peak width.

In this third example of the third implementation, the window vector w captured at step 1120 comprises a single component signal window w. To implement step 1130, the response detector 320-1 measures the amplitude d₁ of an ECAP from a particular fibre type in the signal window w, while the response detector 320-2 measures a characteristic d₂ indicative of the progress of adaptation, such as the peak width of a peak of the ECAP. The completion of adaptation may be determined by comparison of the characteristic d₂ with a target value t₂. Alternatively, the response detector 320-2 may directly detect the presence of adaptation of the same fibre type in the signal window w, so that d₂ is a Boolean value. The controller 116 determines the controller gain K₁ based on d₂, such that K₁ starts off lower than a default value if d₂ indicates that adaptation is present, and switches to the default value once the characteristic d₂ indicates that adaptation is complete. The benefit of this third example of the third implementation is that the primary feedback loop is less responsive at the start of stimulation while adaptation is taking place.

A fourth example of the third implementation is configured to address flexion of the electrode array caused by posture change. The primary feedback loop may be configured to operate in a manner that maintains near-constant recruitment through changes in overall electrode-to-cord distance. One example of such a configuration, referred to as “I-V control”, is described in International Patent Publication no. WO2017/173493, the entire contents of which are herein incorporated by reference. I-V control is predicated on a loop parameter k that captures the relative variation of both stimulation sensitivity and measurement sensitivity with electrode-to-cord distance. However, I-V control with a constant value of the loop parameter k assumes that the distance between the SEC and the cord is always the same as the distance between the MEC and the cord. If the electrode array flexes about a point between the SEC and the MEC, this assumption may be violated.

In this fourth example of the third implementation, the loop parameter k for I-V control according to the primary measured characteristic d₁ is configured to vary depending on the amount of flexion between the SEC and the MEC. Flexion may be monitored by monitoring the sensitivity S at each MEC along the array while keeping the SEC fixed. Non-uniform changes of sensitivity S along the array are indicative of flexion of the array relative to the cord. The controller 116 may repeatedly determine the loop parameter k of I-V control depending on the amount of flexion.

According to a fourth implementation of the multi-response single-stimset CLNS system 1000 and the method 1100, the controller 116 supervises the operation of the primary feedback loop that is driven by the primary measured characteristic d₁ based on one or more of the measured characteristics dⱼ and/or their respective target values tⱼ. For example, the controller 116 may enable and disable the primary feedback loop based on the one or more secondary measured characteristics dⱼ (for j>1), possibly in conjunction with their respective target values tⱼ. In such an example, the controller 116 may determine a Boolean variable indicating the enablement of feedback control by comparing the one or more secondary measured characteristics dⱼ with their respective target values tⱼ. When feedback control is disabled, the multi-response feedback controller 1010 may operate in open-loop mode, in which the stimulus parameters are held constant or made directly adjustable by operation of the remote controller 720.

In a first example of the fourth implementation, the window vector w captured at step 1120 comprises a single component signal window w. To implement step 1130, The response detector 320-1 measures the amplitude d₁ of an ECAP from a particular fibre type in the signal window w, and the response detector 320-2 detects the presence of a late response from the same fibre type in the signal window w. The characteristic d₂ is therefore a Boolean variable. The controller 116 disables the primary feedback loop when a late response is detected, as indicated by the characteristic d₂ being True, and re-enables the primary feedback loop when a late response is no longer detected, as indicated by the characteristic d₂ being False.

In a second example of the fourth implementation, the window vector w captured at step 1120 comprises a single component signal window w. To implement step 1130, the response detector 320-1 measures a primary characteristic d₁ in the signal window w, and the response detector 320-2 measures a secondary characteristic d₂ in the signal window w. The target values t₁ and t₂ are dynamic in that either or both are adjustable by the patient, e.g. by operation of the remote controller 720.

In the primary loop, the multi-response feedback controller 1010 adjusts a stimulus parameter s after each delivered stimulus to maintain the primary characteristic d₁ at the dynamic target value t₁. In the secondary loop, the multi-response feedback controller 1010 adjusts the stimulus parameter s after each delivered stimulus to maintain the secondary characteristic d₂ at the dynamic target value t₂. The multi-response feedback controller 1010 may be implemented using the single-response, single-parameter feedback controller 310 described above. However, only one of the primary feedback loop and the secondary feedback loop is active, i.e. controlling the stimulus parameter s, at any time.

This second example of fourth implementation is particularly useful if a target value tⱼ for a measured characteristic dⱼ is near a saturation region of dⱼ in relation to the stimulus parameter s, i.e. near an upper or lower extremity of the linearly increasing range of the growth curve of dⱼ in relation to s. This can occur if a patient adjusts the target value tⱼ for the measured characteristic dⱼ, to near the saturation region, or a patient changes posture so that a static target value tⱼ moves near the saturation region due to a consequent change in the growth curve for dⱼ. In this circumstance, a single feedback loop maintaining dⱼ at the target value tⱼ by adjusting the stimulus parameter s is likely to be unresponsive while the stimulus parameter s is in the saturation region, since changes in s have relatively little effect on the measured characteristic dⱼ.

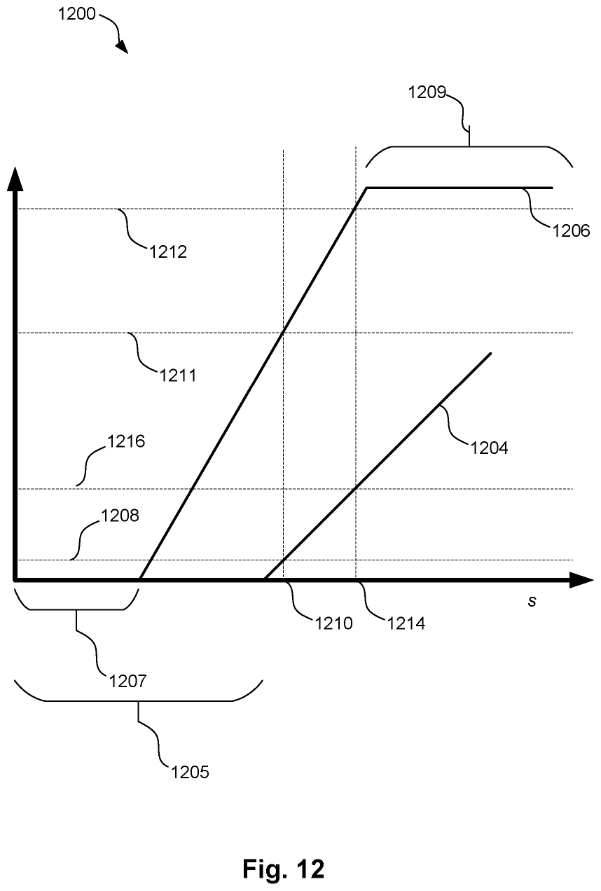

FIG. 12 contains a graph 1200 illustrating this situation. The line 1204 represents a growth curve of the secondary measured characteristic d₂ in relation to a stimulus parameter s. The line 1206 represents a growth curve of the primary measured characteristic d₁ in relation to the stimulus parameter s. The primary growth curve 1206 has a linear region, a saturation region 1209 where the primary growth curve departs from linear increase with respect to s, and a sub-threshold region 1207 in which the primary measured characteristic is not measurable, which is also a form of saturation region. The secondary growth curve 1204 has a linear region and a sub-threshold region 1205 in which the secondary measured characteristic is not measurable.

The stimulus parameter value 1210 is the stimulus parameter value that causes the secondary measured characteristic d₂ to be equal to the secondary target value t₂ 1208. The stimulus parameter value 1210 is near a saturation region 1205 of the secondary growth curve. However, the stimulus parameter value 1210 is not near either of the saturation regions 1207 and 1209 of the primary growth curve 1206. The primary measured characteristic d₁ has a value 1211 at the stimulus parameter value 1210.

Likewise, the stimulus parameter value 1214 is the stimulus parameter value that causes the primary measured characteristic d₁ to be equal to a primary target value t₁ 1212. The stimulus parameter value 1214 is near a saturation region 1209 of the primary growth curve. However, the stimulus parameter value 1214 is not near the saturation region 1205 of the secondary growth curve 1204. The secondary measured characteristic d₂ has a value 1216 at the stimulus parameter value 1214.

In this second example of the fourth implementation, the controller 116 switches the active feedback loop that controls the stimulus parameter s between the primary feedback loop and the secondary feedback loop, depending on where the active measured characteristic d₁ or d₂, or the corresponding target value t₁ or t₂, lies in relation to its corresponding saturation region.

In this second example of the fourth implementation, the primary measured characteristic d₁ is an amplitude of an ECAP from a particular fibre type and has a dynamic target ECAP amplitude t₁. The secondary measured characteristic d₂ is an amplitude of a late response from the same fibre type and has a dynamic target late response amplitude t₂. The stimulus parameter s is the stimulus intensity. The secondary measured characteristic d₂ could alternatively be a different characteristic of the same ECAP that has different saturation regions to d₁ in relation to stimulus intensity s.

To implement step 1140, as long as the secondary feedback loop is the active feedback loop, the multi-response feedback controller 1010 adjusts the stimulus intensity s after each delivered stimulus to maintain the late response amplitude d₂ at the target late response amplitude t₂. Adjustments of the target via the remote controller 720 affect the target late response amplitude t₂. In addition, the controller 116 repeatedly determines a “shadow” target ECAP amplitude t₁ as the ECAP amplitude d₁ at the stimulus intensity at which the late response amplitude d₂ equals the target late response amplitude t₂. In the example of FIG. 12, if the target late response amplitude t₂ is 1208, the “shadow” target ECAP amplitude t₁ is 1211. If the target late response amplitude t₂ is determined to be too close to the saturation region of the growth curve of the late response amplitude d₂, the controller 116 switches the active feedback loop to the primary feedback loop. In this circumstance, the controller 116 promotes the “shadow” target ECAP amplitude t₁ to be the target of the active feedback loop. To implement step 1140, the multi-response feedback controller 1010 adjusts the stimulus intensity s after each delivered stimulus to maintain the ECAP amplitude d₁ at the target ECAP amplitude t₁. Adjustments of the target via the remote controller 720 affect the target ECAP amplitude (1. Meanwhile, the controller 116 repeatedly determines a “shadow” target ECAP amplitude t₂ as the late response amplitude d₂ at the stimulus intensity at which the ECAP amplitude d₁ equals the target ECAP amplitude t₁. In the example of FIG. 12, if the target ECAP amplitude t₁ is 1212, the “shadow” target late response amplitude t₂ is 1216. The switching may be reversed if the target ECAP amplitude t₁ is adjusted to be too close to the upper saturation region of the growth curve of the ECAP amplitude d₁.

The effect of this second example of the fourth implementation if the target late response amplitude gets too close to its lower limit is that the multi-response feedback controller 1010 maintains the late response amplitude at or near the target late response amplitude, but using the ECAP amplitude, which can be measured at stimulation intensities well below the late response threshold, rather than the late response amplitude. The loop response time for stimulus intensities below the late response threshold is thus improved.

This second example of the fourth implementation may be generalised to switching between more than two feedback loops depending on the closeness of the currently active target to a saturation region of its corresponding growth curve.

According to a second aspect of the present technology, a multi-stimset CNLS system (e.g. the multi-stimset CLNS system 900) is configured to measure a characteristic of an evoked neural response to stimuli in each stimset. The multiple evoked neural responses are used to control one or more parameters of the delivered stimuli in each stimset of the multiple stimsets.

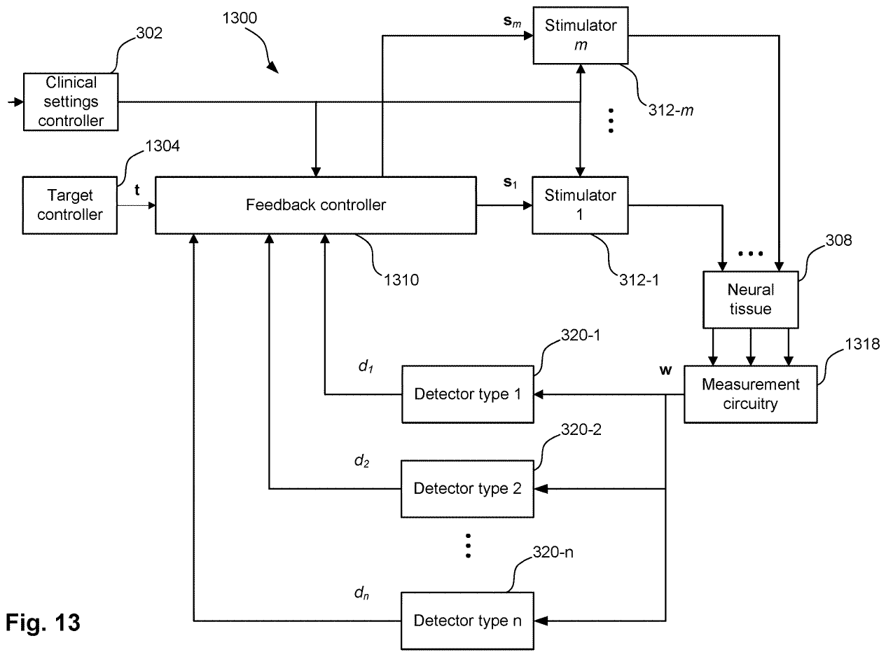

FIG. 13 is a schematic illustrating elements of a multi-response multi-stimset CLNS system 1300, according to the second aspect of the present technology. The multi-response multi-stimset CLNS system 1300 is similar to the multi-response single-stimset CLNS system 1000 of FIG. 10, with like numbers indicating like elements, except that certain elements have been replaced entirely, and new elements have been added. The stimulator 312 has been replaced by m stimulators 312-1, . . . , 312-m, configured to deliver stimuli governed by respective stimulus parameter vectors s₁, . . . , sₘ, via respective SECs. The multi-response feedback controller 1010 has been replaced by the multi-response multi-stimset feedback controller 1310. The multi-response multi-stimset feedback controller 1310 receives and analyses the characteristics d₁, d₂, . . . , dₙ from the respective response detectors 320-1, 320-2, . . . , 320-n to generate the stimulus parameter vectors s₁, . . . , Sₘ. The target controller 1304 feeds a target vector t to the multi-response multi-stimset feedback controller 1310. The measurement circuitry 1318 is the same as the measurement circuitry 1018 of FIG. 10. The clinical settings controller 302 provides the stimulus parameters to the stimulators 312-1, . . . , 312-m that are not under the control of the multi-response multi-stimset feedback controller 1310.

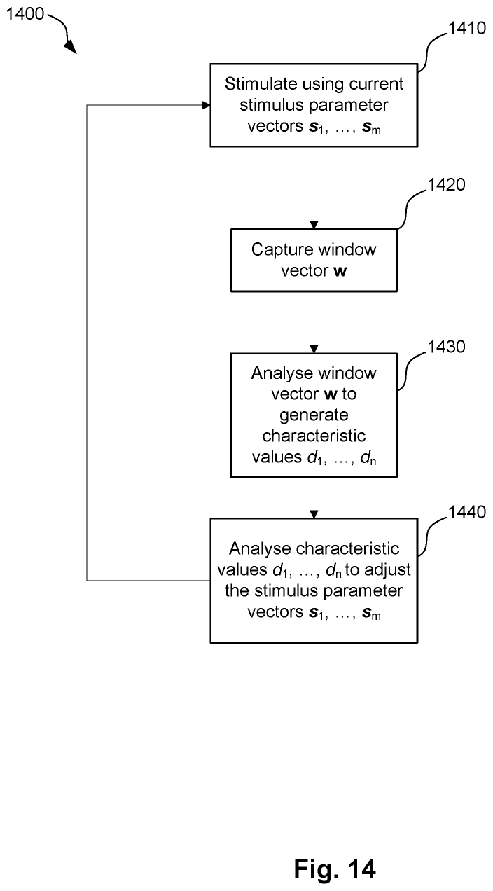

FIG. 14 is a flowchart illustrating a method 1400 of operating a multi-response, multi-stimset feedback loop according to the second aspect of the present technology. The method 1400 may be implemented by the multi-response multi-stimset CLNS system 1300 of FIG. 13. The method 1400 starts at step 1410, at which the stimulators 312-1, . . . , 312-m deliver respective neural stimuli in accordance with the current value of their respective stimulus parameter vectors s₁, . . . , sₘ via their respective SECs. Step 1420 then captures the window vector w subsequent to the delivered stimulus from step 1410. (In some implementations using a single MEC, as described above, the method 1400 may need to loop back from step 1420 to step 1410 at least once to deliver more stimuli until the window vector w is fully populated with signal windows.) Step 1430 uses the neural response detectors 320-1, 320-2, . . . , 320-n to generate the characteristics d₁, d₂, . . . , dₙ from the window vector w. At step 1440, the multi-response multi-stimset feedback controller 1310 analyses the characteristics d₁, d₂, . . . , dₙ from the neural response detectors 320-1, 320-2, . . . , 320-n and the target vector t from the target controller 1304 to adjust the stimulus parameter vectors s₁, . . . , sm. The method 1400 then loops back to step 1410 to deliver the next neural stimulus using the adjusted stimulus parameter vectors s₁, . . . , sₘ.

The method 1400 is specific to the case where all the stimsets are being delivered synchronously, that is, at the same stimulus frequency. A more general method of operating a multi-response, multi-stimset feedback loop according to the second aspect of the present technology may comprise multiple parallel instances of the method 1100, with each instance corresponding to one stimset. Such a method is suitable for the case where the stimsets are being delivered at different stimulus frequencies.

Some implementations of the multi-response multi-stimset CLNS system 1300 and the method 1400 are the same as above-described implementations of the first aspect, with the stimulator 312-1 delivering the stimuli of the applied stimset. The stimuli delivered via the applied stimset stimulator 312-1 at step 1410 evoke the responses whose characteristics d₁, . . . , dₙ are measured by the respective neural response detectors 320-1, 320-2, . . . , 320-n. The stimulus parameter vectors s₁, . . . , sₘ are scalars, equal to the stimulus intensity parameters s₁, . . . , sₘ. To implement step 1440, The multi-response multi-stimset feedback controller 1310 adjusts the stimulus intensity parameter s₁ after each delivered stimulus of the applied stimset to maintain a feedback variable derived from the measured characteristics d₁, . . . , dₙ at its target value t₁ as described above in relation to the first, second, third, and fourth implementations of the first aspect. The multi-response multi-stimset feedback controller 1310 also adjusts the stimulus intensity parameters s₂, . . . , sₘ of the non-applied stimsets in fixed ratio with the applied stimset intensity parameter s₁, as described above in relation to FIG. 9.

In a second implementation of the multi-response multi-stimset CLNS system 1300, each stimset operates as an independent feedback loop according to its own corresponding measured characteristic and target value (so m=n). To implement step 1440, the multi-response multi-stimset feedback controller 1310 adjusts each stimulus intensity parameter vector sⱼ after each delivered stimulus of the corresponding stimset to maintain the measured characteristic dⱼ evoked by that stimset at its corresponding target tⱼ. The multi-response multi-stimset feedback controller 1310 may be implemented as m separate instances of the single-response, single-parameter feedback controller 310 described above.

In a third implementation of the multi-response multi-stimset CLNS system 1300, each stimset operates as a feedback loop according to its own corresponding measured characteristic and target value (so m=n). However, there is interaction between the loops, in that the target value of one feedback loop is modulated by the measured characteristic of another feedback loop. In one example of such an implementation, there are two stimsets (i.e. m=2). Stimset 1 targets A-delta fibres, while stimset 2 targets A-beta fibres. However, stimset 1 targeting A-delta fibres will inevitably recruit a predetermined proportion of A-beta fibres as well, whereas stimset 2 may be configured to target A-beta fibres more or less exclusively. To keep A-beta activation constant overall, the target value t₂ for stimset 2 may be modulated by the measured characteristic d₁ from stimset 1 such that the target value t₂ plus the predetermined proportion of d₁ is equal to a constant.

In a fourth implementation of the of the multi-response multi-stimset CLNS system 1300, there are two stimsets (m=2). The stimuli delivered at step 1410 via the first stimset stimulator 312-1 at a tonic stimulus frequency, e.g. 40 Hz, evoke ECAPs whose amplitudes d₁ are measured by the neural response detector 320-1. The stimuli delivered at step 1410 via the second stimset stimulator 312-2 have a variable stimulus frequency, or equivalently, a variable stimulus period. This is accomplished by setting a component of the stimulus parameter vector s₂ of the second stimset equal to the stimulus period of the second stimset. The stimulus frequency of the second stimset is set generally substantially lower than the (fixed) stimulus frequency of the first stimset. To implement step 1420, the neural response detector 320-2 is configured to measure the amplitude d₂ of the ECAPS evoked by the stimulus delivered via the second stimset stimulator 312-2. The target vector t comprises a target ECAP amplitude t₁ for the first stimset that is near the perception threshold of the patient and a target ECAP amplitude t₂ for the second stimset that is substantially larger than t₁. Even though the target ECAP amplitude t₂ for the second stimset is substantially larger than t₁, the second stimset pulses are generally not perceptible because the stimulus frequency of the second stimset is set generally substantially lower than the stimulus frequency of the first stimset.

To implement step 1440, the multi-response multi-stimset feedback controller 1310 adjusts the stimulus intensity parameter s₁ after each delivered stimulus of the first stimset to maintain the measured ECAP amplitude d₁ evoked by the first stimset at its target ECAP amplitude t₁ as described above. Because the target ECAP amplitude t₁ is near the perception threshold of the patient, the first stimset stimuli are barely perceptible by the patient. The multi-response multi-stimset feedback controller 1310 also adjusts the stimulus intensity parameter of the second stimset in fixed ratio with the first stimset intensity parameter s₁, as described above in relation to FIG. 9. The multi-response multi-stimset feedback controller 1310 also adjusts the stimulus period of the second stimset to maintain the measured ECAP amplitude d₂ evoked by the second stimset at its target ECAP amplitude t₂. For example, if the measured ECAP amplitude d₂ is smaller than the target ECAP amplitude t₂, the multi-response multi-stimset feedback controller 1310 increases the stimulus period of the second stimset. If the measured ECAP amplitude d₂ is larger than the target ECAP amplitude t₂, the multi-response multi-stimset feedback controller 1310 maintains or decreases the stimulus period of the second stimset. This is effective because the measured ECAP amplitude d₂ is dependent on the stimulus period of the second stimset due to the phenomenon of adaptation. A decreasing ECAP amplitude d₂ is indicative of the stimulus period being small enough that adaptation occurs between stimuli (a sign of nervous system integration and hence increased perception). The stimulus period may therefore be increased to reduce the amount of adaptation between stimuli, thereby keeping the second stimset below perception.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

LABEL LIST

| | |
|---|---|
| stimulator | 100 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| pulse generator | 124 |
| electrode selection module | 126 |
| measurement circuitry | 128 |
| system ground | 130 |
| electrode array | 150 |
| biphasic stimulus pulse | 160 |
| neural response | 170 |
| nerve | 180 |
| communications channel | 190 |
| external device | 192 |
| CLNS system | 300 |
| clinical settings controller | 302 |
| target ECAP controller | 304 |
| box | 308 |
| box | 309 |
| single-parameter feedback controller | 310 |
| box | 311 |
| stimulator | 312 |
| stimulator | 312A |
| stimulator | 312B |
| stimulator | 312C |
| stimulator | 312D |
| stimulator | 312-1 |
| stimulator | 312-2 |
| stimulator | 312-m |
| element | 313 |
| measurement circuitry | 318 |
| signal window | 319 |
| ECAP detector | 320 |
| neural response detector | 320-1 |
| neural response detector | 320-2 |
| neural response detector | 320-i |
| neural response detector | 320-j |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP thresholds | 510 |
| ECAP threshold | 512 |
| ECAP target | 520 |
| ECAP | 600 |
| neural stimulation system | 700 |
| neuromodulation device | 710 |
| remote controller | 720 |
| clinical settings transceiver | 730 |
| clinical interface | 740 |
| charger | 750 |
| illustration | 800 |
| stimulus pulse | 810 |
| stimulus pulse | 820 |
| stimulus pulse | 830 |
| stimulus pulse | 840 |
| stimulus pulse | 850 |
| ECAP | 860 |
| multi-stimset CLNS system | 900 |
| multi-response single-stimset | 1000 |
| CLNS system | |
| target controller | 1004 |
| multi-response feedback | 1010 |
| controller | |
| measurement circuitry | 1018 |
| method | 1100 |
| step | 1110 |
| step | 1120 |
| step | 1130 |
| step | 1140 |
| graph | 1200 |
| line | 1204 |
| saturation region | 1205 |
| line | 1206 |
| sub-threshold region | 1207 |
| saturation region | 1209 |
| value | 1210 |
| value | 1211 |
| value | 1214 |
| value | 1216 |
| multi-response multi-stimset | 1300 |
| CLNS system | |
| target controller | 1304 |
| multi-response multi-stimset | 1310 |
| feedback controller | |
| measurement circuitry | 1318 |
| method | 1400 |
| step | 1410 |
| step | 1420 |
| step | 1430 |
| step | 1440 |

1-29. (canceled)

30. An implantable device for controllably delivering neural stimuli, the device comprising:
a stimulus source configured to provide neural stimuli to be delivered according to one of a plurality of stimulation sets to a neural pathway of a patient in order to evoke neural responses from the neural pathway, wherein each stimulation set comprises one or more stimulus electrodes;
measurement circuitry configured to capture signal windows sensed on the neural pathway subsequent to respective neural stimuli, each signal window sensed via one or more measurement electrodes; and
a control unit configured to:
control the stimulus source to provide a first neural stimulus according to a first stimulation set of the plurality of stimulation sets and according to one or more first stimulus parameters;
control the stimulus source to provide a second neural stimulus according to a second stimulation set of the plurality of stimulation sets and according to one or more second stimulus parameters;
measure a first characteristic of a first evoked neural response in a first captured signal window subsequent to the first neural stimulus;
measure a second characteristic of a second evoked neural response in a second captured signal window subsequent to the second neural stimulus;
adjust, using a feedback controller:
the one or more first stimulus parameters so as to maintain the first measured characteristic at or near a first target value, and
the one or more second stimulus parameters so as to maintain the second measured characteristic at or near a second target value; and
adjust the second target value based on the first measured characteristic.

31. The implantable device of claim 30, wherein the control unit is configured to adjust the second target value such that the second target value plus a predetermined proportion of the first measured characteristic is equal to a predetermined constant.

32. An automated method of controllably delivering neural stimuli, the method comprising:
controlling a stimulus source to deliver a first neural stimulus via a first stimulation set of a plurality of stimulation sets according to one or more first stimulus parameters to a neural pathway of a patient in order to evoke a first neural response from the neural pathway;
controlling the stimulus source to deliver a second neural stimulus via a second stimulation set of the plurality of stimulation sets according to one or more second stimulus parameters to a neural pathway of a patient in order to evoke a second neural response from the neural pathway;
capturing a plurality of signal windows sensed on the neural pathway subsequent to the delivered neural stimuli;
measuring a first characteristic of the first evoked neural response in a first captured signal window of the plurality of signal windows subsequent to the first neural stimulus;
measuring a second characteristic of the second evoked neural response in a second captured signal window of the plurality of signal windows subsequent to the second neural stimulus;
adjusting the one or more first stimulus parameters so as to maintain the first measured characteristic at or near a first target value; and
adjusting the one or more second stimulus parameters so as to maintain the second measured characteristic at or near a second target value; and
adjusting the second target value based on the first measured characteristic.

33. The method of claim 32, wherein adjusting the second target value comprises adjusting the second target value such that the second target value plus a predetermined proportion of the first measured characteristic is equal to a predetermined constant.

34. An implantable device for controllably delivering neural stimuli, the device comprising:
a stimulus source configured to provide neural stimuli to be delivered via one of a plurality of stimulation sets to a neural pathway of a patient in order to evoke neural responses from the neural pathway, wherein each stimulation set comprises one or more stimulus electrodes;
measurement circuitry configured to capture signal windows sensed on the neural pathway subsequent to respective neural stimuli, each signal window sensed via one or more measurement electrodes; and
a control unit configured to:
control the stimulus source to provide a first neural stimulus according to a first stimulation set of the plurality of stimulation sets and according to one or more first stimulus parameters;
control the stimulus source to provide a second neural stimulus according to a second stimulation set of the plurality of stimulation sets and according to one or more second stimulus parameters;
measure a first characteristic of a first evoked neural response in a first captured signal window subsequent to the first neural stimulus;
measure a second characteristic of a second evoked neural response in a second captured signal window subsequent to the second neural stimulus; and
adjust, using a feedback controller:
the one or more first stimulus parameters so as to maintain the first measured characteristic at or near a first target value,
a second stimulus parameter of the one or more second stimulus parameters so as to maintain the second measured characteristic at or near a second target value, and
adjust a further second stimulus parameter of the one or more second stimulus parameters based on the adjustment to the one or more first stimulus parameters.

35. The implantable device of claim 34, wherein the second stimulus parameter is a stimulus period.

36. The implantable device of claim 35, wherein the further second stimulus parameter is stimulus intensity.

37. An automated method of controllably delivering neural stimuli, the method comprising:
controlling a stimulus source to deliver a first neural stimulus via a first stimulation set of a plurality of stimulation sets according to one or more first stimulus parameters to a neural pathway of a patient in order to evoke a first neural response from the neural pathway;
controlling the stimulus source to deliver a second neural stimulus via a second stimulation set of the plurality of stimulation sets according to one or more second stimulus parameters to a neural pathway of a patient in order to evoke a second neural response from the neural pathway;
capturing a plurality of signal windows sensed on the neural pathway subsequent to the delivered neural stimuli;
measuring a first characteristic of the first evoked neural response in a first captured signal window of the plurality of signal windows subsequent to the first neural stimulus;
measuring a second characteristic of the second evoked neural response in a second captured signal window of the plurality of signal windows subsequent to the second neural stimulus;
adjusting the one or more first stimulus parameters so as to maintain the first measured characteristic at or near a first target value;
adjusting a first stimulus parameter of the one or more second stimulus parameters so as to maintain the second measured characteristic at or near a second target value; and
adjusting a further second stimulus parameter of the one or more second stimulus parameters based on the adjustment to the one or more first stimulus parameters.

38. The method of claim 37, wherein the second stimulus parameter is a stimulus period.

39. The method of claim 37, wherein the further second stimulus parameter is stimulus intensity.

40-51. (canceled)
